(19) United States
(12) Patent Application Publication
(10) Pub. No.: US 2008/0319369 A1
(43) Pub. Date: Dec. 25, 2008

(54) Systems and Methods for Removing Fluid and Atherogenic, Thrombotic, and Inflammatory Markers From Blood

(76) Inventors: George Trutza, East Greenwich, RI (US); Robert A. Rabiner, Tiverton, RI (US)

Correspondence Address: GREENBERG TRAURIG, LLP, ONE INTERNATIONAL PLACE, 20th FL, ATTN: PATENT ADMINISTRATOR, BOSTON, MA, 02110, US

(21) Appl. No.: 12/143,296

(22) Filed: Jun. 20, 2008

Related U.S. Application Data
(60) Provisional application No. 60/936,871, filed on Jun. 22, 2007.

(51) Int. Cl. A61M 1/34

(52) U.S. Cl. 604/6.01; 604/6.09

(57) ABSTRACT

Systems and methods for removing fluid and atherogenic, thrombotic and inflammatory (ATI) markers from blood are disclosed. A system for removing fluid and atherogenic, thrombotic and inflammatory markers from blood includes an ultrafiltration module for removing fluid from blood drawn from a patient; and an apheresis module for removing atherogenic, thrombotic, and inflammatory markers from the blood, wherein the ultrafiltration module and the apheresis module form a continuous closed-loop extracorporeal blood circuit.

### RELATED APPLICATIONS

This application claims the benefit of U.S. Provisional Application Ser. No. 60/936,871, filed on Jun. 22, 2007, the entirety of which is hereby incorporated herein by reference for the teachings therein.

### FIELD

The embodiments disclosed herein relate to the treatment of congestive heart failure, and more particularly to systems and methods to remove fluid as well as atherogenic, thrombotic and inflammatory markers from blood.

### BACKGROUND

Congestive Heart Failure (CHF) is a life-threatening condition in which the heart can no longer pump enough blood to the rest of the body. CHF is typically brought on by an underlying heart or blood vessel problem, often a combination of different problems. The most common cause of heart failure is Coronary Artery Disease (CAD). An individual with an increased blood viscosity has a higher chance of developing CAD. Increased levels of certain plasma proteins, particularly fibrinogen, immunoglobulins, and lipoproteins, often play a role in increasing the bloods viscosity. The increase in blood viscosity strains the heart, and the heart needs to work harder to pump and circulate the blood, which may cause CHF.

As CHF develops, blood does not move efficiently through the circulatory system and starts to back up, increasing the pressure in the blood vessels and forcing fluid from the blood vessels into body tissues. When the left side of the heart starts to fail, fluid collects in the lungs. This extra fluid in the lungs (congestion) makes it more difficult for the airways to expand as a person inhales. When the right side of the heart starts to fail, fluid collects in the feet and lower legs. As the heart failure becomes worse, the upper legs swell and eventually the abdomen collects fluid.

The presence of certain atherogenic, thrombotic, and inflammatory (ATI) markers in the plasma often results in heart failure. This in turn, causes fluid-overload throughout the body. The fluid-overload and associated clinical symptoms resulting from these physiologic changes are the predominant cause for excessive hospital admissions, diminishing quality of life and overwhelming costs to the health care system due to heart failure.

There is a need for a system for the treatment of CHF which is able to remove ATI markers and excess fluid from blood. For patients suffering with CHF, such a system could lower the blood viscosity, increase the blood circulation, reduce the strain on the heart and reduce excess fluid in blood.

### BRIEF DESCRIPTION OF THE DRAWINGS

The presently disclosed embodiments will be further explained with reference to the attached drawings, wherein like structures are referred to by like numerals throughout the several views. The drawings shown are not necessarily to scale, with emphasis instead generally being placed upon illustrating the principles of the presently disclosed embodiments.

FIG. 1 is a flow chart illustrating a procedure for removing atherogenic, thrombotic and inflammatory (ATI) markers and excess fluid from whole blood components using the presently disclosed embodiments.

FIG. 2 is a flow chart illustrating a procedure for removing excess fluid and atherogenic, thrombotic and inflammatory (ATI) markers from whole blood components using the presently disclosed embodiments.

FIG. 3 is a flow chart illustrating a procedure for removing atherogenic, thrombotic and inflammatory (ATI) markers and excess fluid from whole blood components using the presently disclosed embodiments.

FIG. 4 is a schematic diagram illustrating an embodiment of a system for the removal of atherogenic, thrombotic and inflammatory (ATI) markers and excess fluid from whole blood components using the procedure described in FIG. 1.

FIG. 5 is a schematic diagram illustrating an embodiment of a system for the removal of excess fluid and atherogenic, thrombotic and inflammatory (ATI) markers from whole blood components using the procedure described in FIG. 2.

FIG. 6 is a schematic diagram illustrating an embodiment of a system for the removal of excess fluid and atherogenic, thrombotic and inflammatory (ATI) markers from whole blood components using the procedure described in FIG. 3.

While the above-identified drawings set forth presently disclosed embodiments, other embodiments are also contemplated, as noted in the discussion. This disclosure presents illustrative embodiments by way of representation and not limitation. Numerous other modifications and embodiments may be devised by those skilled in the art which fall within the scope and spirit of the principles of the presently disclosed embodiments.

### DETAILED DESCRIPTION

Systems and methods for the removal of excess fluid and atherogenic, thrombotic and inflammatory (ATI) markers from blood are disclosed herein. “Fluid” as used herein includes salt and water. “Blood” as used herein includes plasma and formed elements. “Formed elements” as used herein includes erythrocytes (red blood cells), leukocytes (white blood cells), and the thrombocytes (platelets). The systems and methods disclosed herein safely, simply, and precisely remove excess fluid from blood. The systems and methods also selectively remove ATI markers and cardiovascular risk markers from blood and treat patients with congestive heart failure (CHF). CHF may result from narrowed arteries that supply blood to the heart muscle (Coronary Artery Disease), one or more previous heart attacks or myocardial infarctions, one or more previous strokes, high blood pressure, heart valve disease due to past rheumatic fever or other causes, primary disease of the heart muscle (cardiomyopathy), congenital heart defects, and infection of the heart valves and/or heart muscle (endocarditis and/or myocarditis).

Atherogenic, thrombotic and inflammatory markers include, but are not limited to, total cholesterol, low-density lipoprotein cholesterol (LDL-C) or “bad cholesterol”, Very Low Density Lipoproteins (VLDL), lipoprotein [a] (Lp[a]), triglycerides, fibrinogen, thrombin, Factor V, Factor VII, Von Willebrand factor (vWF), ATP III, C-reactive protein (CRP), plasma viscosity (PV) and erythrocyte aggregation (EA).

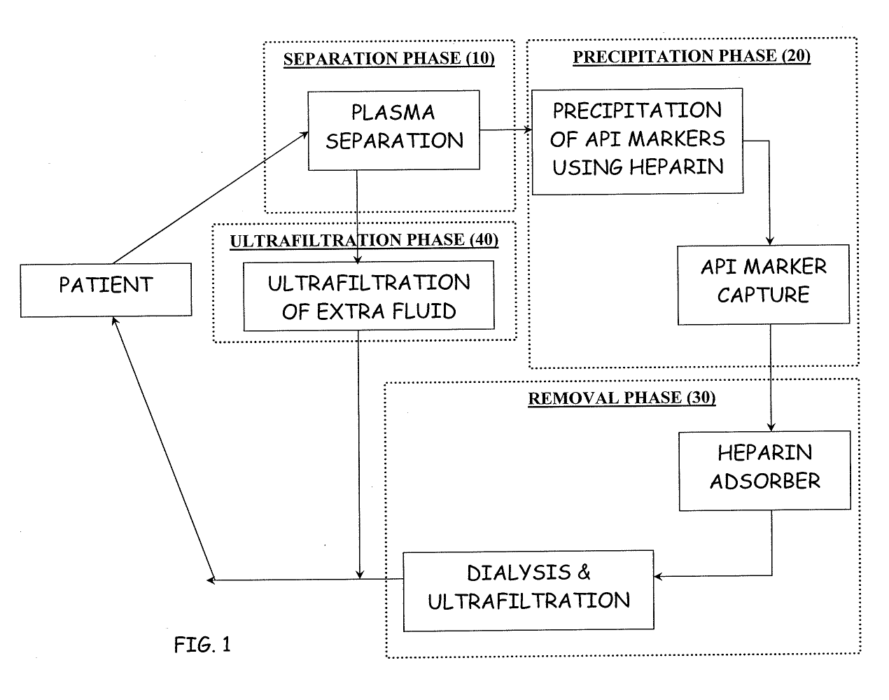

FIG. 1 shows a flow chart illustrating a procedure for removing excess fluid and ATI markers from blood using the presently disclosed embodiments. The procedure generally includes four phases: a separation phase 10, a precipitation phase 20, a removal phase 30 and an ultrafiltration phase 40. During the separation phase 10, the blood is separated into formed elements and plasma. In this phase, blood is drawn from a vein in a patient and passed into a capillary plasma filter for plasma separation. Blood flow is controlled by a pump. The separation phase is a continuous phase which means that the blood processing and the return of blood to the patient are done concurrently.

In the ultrafiltration phase 40, the formed elements are passed through a filter which separates excess fluid from the formed elements, yielding fluid-depleted formed elements. The excess fluid is captured in a collection bag and can be measured.

During the precipitation phase 20, a solution containing heparin and an acetate buffer is added to the plasma to lower the pH level. The heparin adheres to the ATI markers, causing them to precipitate. A small-pored filter captures the precipitate and the ATI-reduced plasma passes through this filter.

During the removal phase 30, the ATI-reduced plasma passes through filters that remove excess heparin and adjust the volume of the plasma. Those skilled in the art will recognize that the precipitation phase and the removal phase are performed at least one time. In an embodiment, the precipitation phase and the removal phase are performed at least two times. The ATI-reduced plasma is then recombined with the fluid-depleted formed elements and returned to the patient's circulation through a different vein. In an embodiment, a single procedure treats between about 2.5 and about 3.0 liters of plasma. In an embodiment, at any given time during the treatment, the total volume of formed elements does not exceed about 150 mL and the total volume of plasma does not exceed about 400 mL. Following the procedure, the patient's ATI marker levels are dramatically reduced. Typical reductions in various ATI markers are shown for example, in Table 1 below.

TABLE 1
Acute Reductions of ATI markers

| ATI Marker | Reduction* |
|---|---|
| Total Cholesterol | P569 52% |
| LDL Cholesterol | P569 56% |
| VLDL | P569 52% |
| Lp(a) | P569 55% |
| Triglycerides | P569 50% |
| Fribrinogen | P569 56% |
| Thrombin | P569 55% |
| Factor V | P569 57% |
| Factor VII | P569 35% |
| v W F | P569 56% |
| ATP III | P569 25% |
| CRP | P569 65% |
| PV | P569 14% |
| EA | P569 60% |

*Reductions are approximate

Hemorheology, in particular plasma viscosity (PV) and erythrocyte aggregation (EA) have been linked to the development of atherosclerosis and cardiovascular complications. As shown in Table 1, after a single treatment with the system of the presently disclosed embodiments, PV may be reduced by about 14% and EA may be reduced by about 60%. The reduction in PV and EA improves endothelial function, thereby improving blood circulation and reducing the stress on the heart.

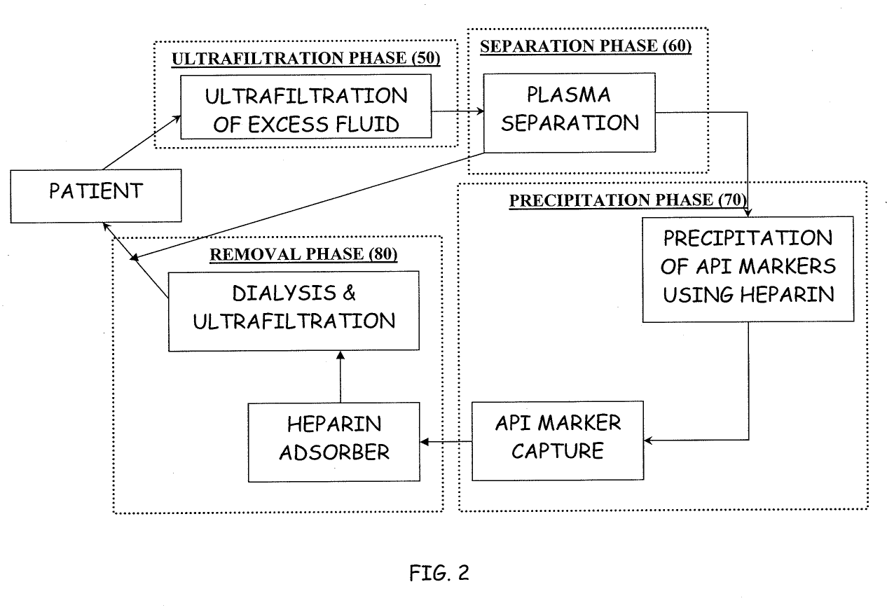

FIG. 2 shows a flow chart illustrating a procedure for removing excess fluid and ATI markers from a patient's blood using the presently disclosed embodiments. In this embodiment, the ultrafiltration phase 50 is done prior to the plasma separation. The procedure shown in FIG. 2 begins with the drawing of blood from a vein in a patient. Blood flow is controlled by a pump. In the ultrafiltration phase 50, blood drawn from the patient is passed through a filter to separate excess fluid from the blood. A collection bag holds the excess fluid and allows measurement of the amount of fluid removed. The fluid-depleted blood is then sent to a capillary plasma filter.

In the separation phase 60, the fluid-depleted blood is separated into formed elements and plasma by passage through the capillary plasma filter. The separation phase 60 is a continuous phase which means that the blood processing and the return of blood to the patient are done concurrently.

During the precipitation phase 70, a solution containing heparin and an acetate buffer is added to the plasma to lower the pH level. The heparin adheres to ATI markers, causing them to precipitate. A small-pored filter captures the precipitate and the ATI-reduced plasma passes through this filter.

During the removal phase 80, the ATI-reduced plasma passes through filters that remove excess heparin and adjust the volume of the plasma. Those skilled in the art will recognize that the precipitation phase and the removal phase are performed at least one time. In an embodiment, the precipitation phase and the removal phase are performed at least two times. The ATI-reduced plasma is then recombined with the fluid-depleted formed elements and returned to the patient's circulation through a different vein. In an embodiment, a single procedure treats between about 2.5 and about 3.0 liters of plasma. In an embodiment, at any given time during the treatment, the total volume of formed elements does not exceed about 150 mL and the total volume of plasma does not exceed about 400 mL. Following the procedure, the patient's ATI marker levels are dramatically reduced. Typical reductions in various ATI markers are shown for example, in Table 1 above.

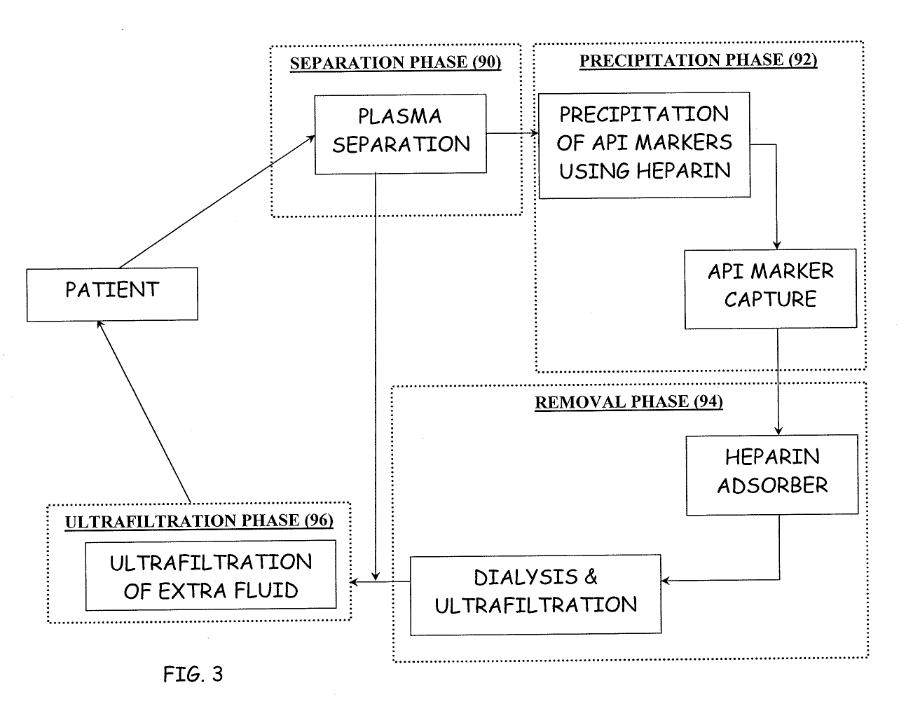

FIG. 3 shows a flow chart illustrating a procedure for removing excess fluid and ATI markers from a patient's blood using the presently disclosed embodiments. In this embodiment, the ultrafiltration phase 96 is performed after the ATI markers have been removed from the plasma. The procedure shown in FIG. 3 begins with the drawing of blood from a vein in a patient. Blood flow is controlled by a pump. In the separation phase 90, the blood is separated into formed elements and plasma by passage through a capillary plasma filter. The separation phase 90 is a continuous phase which means that the blood processing and the return of blood to the patient are done concurrently.

During the precipitation phase 92, a solution containing heparin and an acetate buffer is added to the plasma to lower the pH level. The heparin adheres to the ATI markers, causing them to precipitate. A small-pored filter captures the precipitate and the ATI-reduced plasma passes through this filter.

During the removal phase 94, the ATI-reduced plasma passes through filters that remove excess heparin and correct the volume of liquid plasma. Those skilled in the art will recognize that the precipitation phase and the removal phase are performed at least one time. In an embodiment, the precipitation phase and the removal phase are performed at least two times. The ATI-reduced plasma is then recombined with the fluid-depleted formed elements and is passed through a filter to separate excess fluid from the ATI-reduced fluid-depleted blood, the ultrafiltration phase 96. A collection bag holds the excess fluid and allows measurement of the amount of fluid removed. The ATI-reduced fluid-depleted blood is then returned to the patient's circulation through a different vein. In an embodiment, a single procedure treats between about 2.5 and about 3.0 liters of plasma. In an embodiment, at any given time during the treatment, the total volume of formed elements does not exceed about 150 mL and the total volume of plasma does not exceed about 400 mL. Following the procedure, the patient's ATI marker levels are dramatically reduced. Typical reductions in various ATI markers are shown for example, in Table 1 above.

The systems disclosed herein are extracorporeal blood circuits having an apheresis module and an ultrafiltration module. In an embodiment, the blood passage through the circuit is air free, and has smooth passage walls promoting continuous and uniform flow of the blood through the circuit. Generally, the extracorporeal blood circuit is comprised of blood pumps, constituent tubing, pressure sensors, air sensors and various filters. The apheresis module is based on heparin precipitation using a heparin extracorporeal low-density lipoprotein precipitation (HELP) process, as described by D. Seidel, Therapeutische Rundschau, 47 (1990), pp. 514-519, which is hereby incorporated by reference, for the removal of ATI markers in treated plasma.

The ultrafiltration module relies on a membrane filter to remove excess fluid from blood at a controlled rate. A roller pump placed between the filter and an ultrafiltrate collection bag controls the flow of ultrafiltrate (fluid) through the filter. When the pump is slowed down ultrafiltrate flow is retarded, pressure gradient across the membrane is reduced and ultrafiltration is slowed to a desired level. Alternatively, if the pump RPM is increased, the flow of ultrafiltrate is accelerated. Negative pressure may be developed by the pump to actively suck the ultrafiltrate across the membrane filter. The ultrafiltrate is collected into a sealed bag connected by a tube to the ultrafiltrate collection chamber of the filter casing. During a treatment the bag is gradually filled up with fluid. When the bag is full, the pressure in the bag will start to rise until it is equal to the average pressure of blood inside the filter capillaries. Although some circulation of fluid is still possible in and out of fibers the net loss of fluid is about zero. Until the bag is emptied, removal of fluid is severely slowed. The systems disclosed herein are continuous and closed-loop, which means that the blood processing and the return of blood to the patient are done concurrently. For simplicity purposes, pumps, sensors, and constituent tubing are not shown in the following figures.

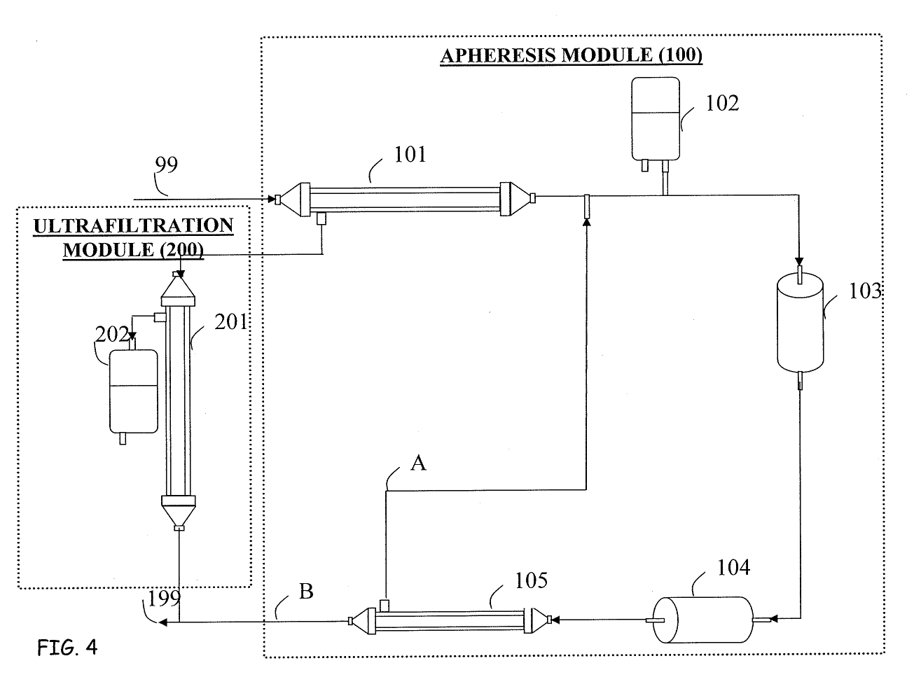

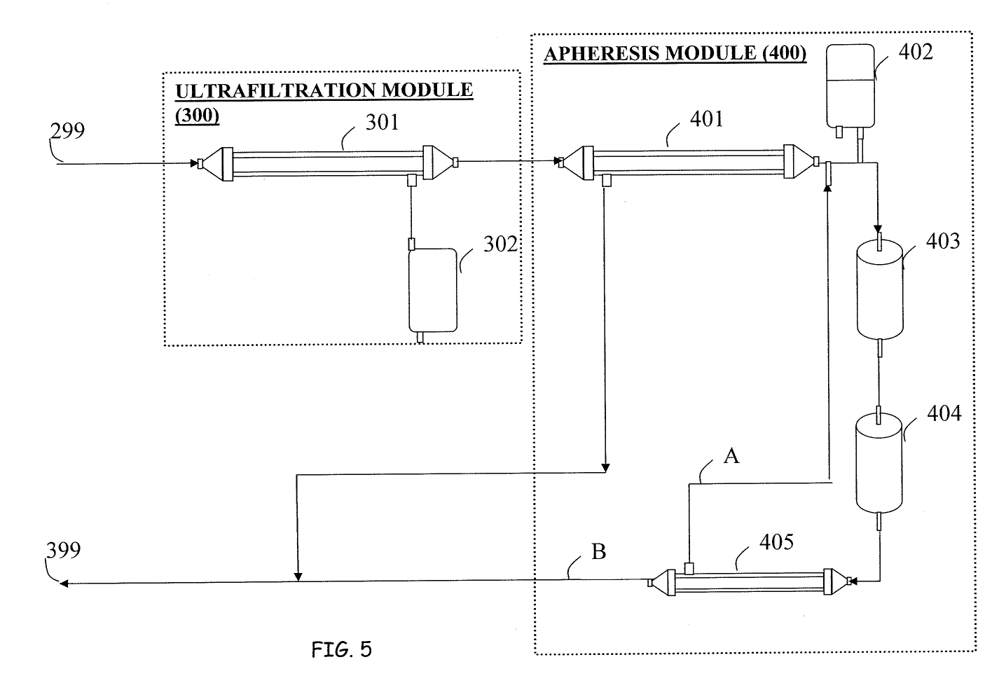

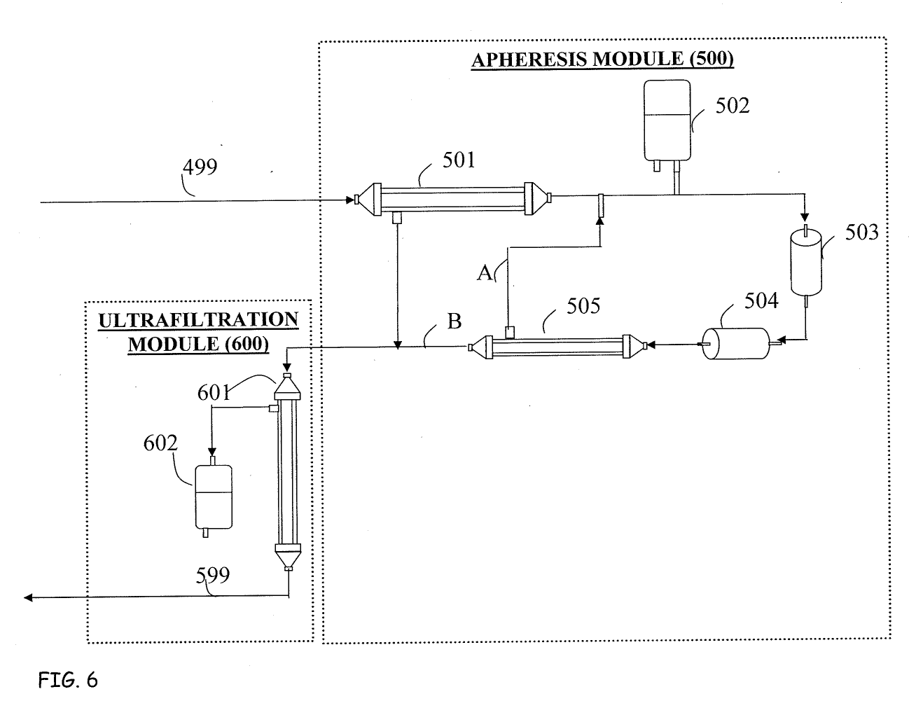

FIG. 4, FIG. 5, and FIG. 6 show systems having modules that perform key functions of the presently disclosed embodiments. In the systems, both apheresis (the removal of ATI markers) and ultrafiltration (the removal of fluid) from blood are taking place in one continuous and closed-loop process. Those skilled in the art will recognize that the placement of the modules may vary and still be within the scope of the presently disclosed embodiments.

FIG. 4 shows a system for removing ATI markers and fluid from blood. As shown in FIG. 4, the process begins with the continuous withdrawal of blood 99 from a catheter in a peripheral or central vein, typically in a patient's arm, which is then fed to a capillary plasma filter 101 for separation. The capillary plasma filter separates the formed elements from the plasma. The formed elements are then passed through an ultra-filter 201, which is part of the ultrafiltration module 200. The ultra-filter 201 removes excess fluid from the formed elements. In an embodiment, the ultra-filter 201 provides a smooth flow path for the formed elements through the filter passages. In an embodiment, a membrane surface area of about 0.1 m² provides sufficient fluid removal during operation of the extracorporeal circuit. A smooth flow path is achieved by making the ultra-filter 201 long and thin. In an embodiment, the ultra-filter 201 has an effective length of about 22.5 cm and a fiber bundle diameter of about 1.2 cm.

A parameter for the characterization of the flow characteristic of an extracorporeal circuit or of the circuits components is the residence time. This method of characterizing an extracorporeal has been described, e.g., by Cooney D O, Infantolino W, Kane R. in “Comparative Studies of Hemoperfusion Devices.” For a passive extracorporeal device, the total residence time of the blood in the device should be minimized to reduce the potential for clotting. The rate at which fluid is extracted from the formed elements is adjustable. The extraction rate may be adjusted to match the natural rate of excess fluid returning from tissues and entering the blood circulatory. This adjustability allows a medical practitioner to specify the amount of fluid that needs to be removed on a patient-to-patient basis. In an embodiment, the extraction rate is about 100 to about 500 mL/hour. In an embodiment, the fluid extracted from the blood is between about 20% to about 30% of the blood volume. With this extraction rate, the amount of blood removed from a peripheral vein is less than about 2% of the total cardiac output. In addition, at this extraction rate, the ultrafiltrate flow may be up to about 1 L/hour. In an embodiment, the extraction rate is about 0.1 liter/hour. At a blood flow rate of about 60 mL/min, the amount of fluid extracted may be about 12 mL/min (or 720 mL/hour). In an embodiment, the extraction rate is about 500 mL/hour. The total amount of fluid being removed is measured by collecting the excess fluid in a collection bag 202.

After capillary plasma separation, the plasma is mixed with a mixture 102 of heparin and buffer, for example, sodium acetate buffer. In an embodiment, the sodium acetate buffer and the heparin are present in equivalent parts by volume. In an embodiment the sodium acetate buffer is at a pH of about 4.85 and the heparin has a concentration of about 100 IU/ml. At a plasma reaction pH of about 5.12, ATI marker complexes present in the plasma/buffer suspension are precipitated. In an embodiment, the plasma reaction pH ranges from a pH of about 5.05 to a pH of about 5.25. The plasma/buffer suspension containing the precipitated ATI marker complexes is then pumped to and captured by a precipitate filter 103. A heparin adsorber 104 removes all excess heparin in the plasma/buffer suspension. Before being returned to the patient, the ATI-reduced plasma is brought into a physiologic state. This is achieved by bicarbonate dialysis (pH correction) and subsequent ultrafiltration (volume correction) using an ultra-filter 105. The ultra-filter 105 also removes any excess sodium acetate from the ATI-reduced plasma.

In an embodiment, the ATI-reduced plasma is then recombined with the fluid-depleted formed elements (Line B) and returned to the patient's circulation through a different vein. In an embodiment, the ATI-reduced plasma loops back to the apheresis module 100 so that the ATI-reduced plasma is again mixed with the heparin and buffer 102 mixture (Line A) and continues through the rest of the apheresis module 100. In an embodiment, the ATI-reduced plasma is looped through the apheresis module 100 two times. In an embodiment, the ATI-reduced plasma is looped through the apheresis module 100 four or more times. In an embodiment, the apheresis module 100 removes the ATI markers from the plasma in a period of about 1.5 hours to about four hours. In an embodiment, the ultrafiltration module 200 removes excess fluid from the cellular components for a time period about eight hours.

FIG. 5 shows a system for removing ATI markers and fluid from blood. As shown in FIG. 5, the process begins with the continuous withdrawal of blood 299 from a peripheral or central vein, typically in a patient's arm, which is then fed through an ultra-filter 301, which is part of the ultrafiltration module 300. The ultra-filter 301 removes excess fluid from the blood. The rate at which fluid is extracted from the blood is adjustable. The extraction rate may be adjusted to match the natural rate of excess fluid returning from tissues and entering the blood circulatory. This feature allows a medical practitioner to specify the amount of fluid that needs to be removed on a patient-to-patient basis. The total amount of fluid being removed is measured by collecting the excess fluid in a collection bag 302.

The fluid-depleted blood is then fed to a capillary plasma filter 401 for separation. The capillary plasma filter 401 separates the formed elements from the plasma. The plasma is mixed with a mixture 402 of heparin and buffer, for example, sodium acetate buffer. In an embodiment, the sodium acetate buffer and the heparin are present in equivalent parts by volume. In an embodiment the sodium acetate buffer is at a pH of about 4.85 and the heparin has a concentration of about 100 IU/ml. At a plasma reaction pH of about 5.12, ATI marker complexes present in the plasma/buffer suspension are precipitated. In an embodiment, the plasma reaction pH ranges from a pH of about 5.05 to a pH of about 5.25. The plasma/buffer suspension containing the precipitated ATI marker complexes is then pumped to and captured by a precipitate filter 403. A heparin adsorber 404 removes all excess heparin in the plasma/buffer suspension. Before being returned, the ATI-reduced plasma must first be brought into a physiologic state. This is achieved by bicarbonate dialysis (pH correction) and subsequent ultrafiltration (volume correction) using an ultra-filter 405. The ultra-filter 405 also removes any excess sodium acetate from the ATI-reduced plasma.

In an embodiment, the ATI-reduced plasma is then recombined with the fluid-depleted formed elements (Line B) and returned to the patient's circulation through a different vein. In an embodiment, the ATI-reduced plasma loops back to the apheresis module 400 (Line A) so that the ATI-reduced plasma is again mixed with the heparin and buffer 402 mixture and continues through the rest of the apheresis module 400. In an embodiment, the ATI-reduced plasma is looped through the apheresis module 400 two times. In an embodiment, the ATI-reduced plasma is looped through the apheresis module 400 four or more times. In an embodiment, the apheresis module 400 removes the ATI markers from the plasma in a period of about 1.5 hours to about four hours. In an embodiment, the ultrafiltration module 300 removes excess fluid from the formed elements for a time period of about eight hours.

FIG. 6 shows a system for removing ATI markers and fluid from blood. As shown in FIG. 6, the process begins with the continuous withdrawal of blood 499 from a peripheral or central vein, typically in a patient's arm, which is then fed to a capillary plasma filter 501 for separation. The capillary plasma filter 501 separates the formed elements from the plasma. After capillary plasma separation, the plasma is mixed with a mixture 502 of heparin and buffer, for example, sodium acetate buffer. In an embodiment, the sodium acetate buffer and the heparin are present in equivalent parts by volume. In an embodiment the sodium acetate buffer is at a pH of about 4.85 and the heparin has a concentration of about 100 IU/ml. At a plasma reaction pH of about 5.12, ATI marker complexes present in the plasma/buffer suspension are precipitated. In an embodiment, the plasma reaction pH ranges from a pH of about 5.05 to a pH of about 5.25. The plasma/buffer suspension containing the precipitated ATI marker complexes is then pumped to and captured by a precipitate filter 503. A heparin adsorber 504 removes all excess heparin in the plasma/buffer suspension. The ATI-reduced plasma is then brought into a physiologic state. This is achieved by bicarbonate dialysis (pH correction) and subsequent ultrafiltration (volume correction) using an ultra-filter 505. The ultra-filter 505 also removes any excess sodium acetate from the ATI-reduced plasma.

The ATI-reduced plasma and the formed elements that have been separated using the capillary plasma filter 501 are then re-combined (Line B) and passed through an ultra-filter 601, which is part of the ultrafiltration module 600. The ultra-filter 601 removes excess fluid from the re-combined blood. In an embodiment, the ultra-filter 601 provides a smooth flow path for the blood through the filter passages. In an embodiment, a membrane surface area of about 0.1 m² provides sufficient fluid removal during operation of the extracorporeal circuit. A smooth flow path is achieved by making the ultra-filter 601 long and thin. In an embodiment, the ultra-filter 601 has an effective length of about 22.5 cm and a fiber bundle diameter of about 1.2 cm.

The rate at which fluid is extracted from the re-combined blood is adjustable. The extraction rate is adjustable to match the natural rate of excess fluid returning from tissues and entering the blood circulatory. In an embodiment, the extraction rate is about 100 to about 500 mL/hour. In an embodiment, the fluid extracted from the blood is between about 20% to about 30% of the blood volume. At this extraction rate, the amount of blood removed from a peripheral vein is less than about 2% of the total cardiac output. In addition, at this extraction rate, the ultrafiltrate flow may be as much as about 1 L/hour. In an embodiment, the extraction rate is about 0.1 liter/hour. At a blood flow rate of about 60 mL/min, the amount of fluid extracted may be about 12 mL/min (or 720 mL/hour). It may be possible to extract about 500 mL/hour of excess fluid from the cellular components withdrawn and returned into a peripheral vein. This feature allows a medical practitioner to specify the amount of fluid that needs to be removed on a patient-to-patient basis.

The total amount of fluid being removed is measured by collecting the excess fluid in a collection bag 602. The ATI-reduced fluid-depleted blood is then returned to the patient's circulation through a different vein 599. In an embodiment, the ATI-reduced plasma loops back to the apheresis module 500 (Line A) so that the ATI-reduced plasma is again mixed with the heparin and buffer 502 mixture and continues through the rest of the apheresis module 500. In an embodiment, the ATI-reduced plasma is looped through the apheresis module 500 two times. In an embodiment, the ATI-reduced plasma is looped through the apheresis module 500 four or more times. In an embodiment, the apheresis module 500 removes the ATI markers from the plasma in a period of about 1.5 hours to about four hours. In an embodiment, the ultrafiltration module 600 removes excess fluid from the formed elements in about eight hours. The systems disclosed herein treat a fluid overloaded patient. A patient undergoes treatment while in bed or sitting in a chair while the patient is conscious or asleep. In an embodiment, two relatively standard needles are introduced into suitable peripheral veins (on the same or different arms) for the withdrawal and return of the blood. This procedure is similar to blood draw or IV therapy. Needles are attached to tubing which is secured to the skin.

The ultrafiltration module includes the ultra-filter, tubes, pressure sensors, pumps, and the ultrafiltrate collection bag. Ultrafiltration modules for the removal of excess fluid are known in the art where blood is continuously withdrawn from, processed and returned into the same or different vein in the patient's arm. Examples of apparatus for the removal of excess fluid in the blood are disclosed in U.S. Pat. No. 6,890,315 entitled “Method and apparatus for vein fluid removal in heart failure.”

The apheresis module includes the capillary plasma filter, buffer/heparin bag, precipitate filter, heparin adsorber, ultra-filter (for dialysis and ultrafiltration), tubes, pressure sensors and pumps. Heparin Extracorporeal Low-density Lipoprotein Precipitation (HELP) apheresis for the removal of various ATI markers are known in the art, as described by D. Seidel, Therapeutische Rundschau, 47 (1990), pp. 514-519. The various tubes, sensors, filters, buffer/heparin bag and collection bag are sterile and are not be reused.

Preferably, the ATI markers are depleted for a period of time and to a level that is effective to produce an improvement in a measurable endpoint or clinical improvement in a disease associated with relatively elevated levels of ATI markers, such as certain chronic, age-related, degenerative, atherogenic, thrombotic or inflammatory diseases; especially those associated with causing disturbances of blood rheology or microcirculatory impairment.

The systems and methods of the presently disclosed embodiments produce a decreased concentration of ATI markers and a decrease quantity of fluid in the treated patient. A single treatment session may be effective, although several treatment sessions per week may be necessary to achieve the desired effects. The methods produce a clinically observable improvement in a disorder characterized by elevated levels of ATI markers in a patient.

A system for removing fluid and atherogenic, thrombotic and inflammatory markers from blood includes an ultrafiltration module for removing fluid from blood drawn from a patient; and an apheresis module for removing atherogenic, thrombotic, and inflammatory markers from the blood, wherein the ultrafiltration module and the apheresis module form a continuous closed-loop extracorporeal blood circuit.

A method for removing fluid and atherogenic, thrombotic and inflammatory markers from blood includes withdrawing blood from a catheter in a vein of a patient; passing the blood through a plasma filter that separates the blood into formed elements and plasma; mixing the plasma with a heparin/buffer solution which causes atherogenic, thrombotic, and inflammatory markers in the plasma to form a precipitate, resulting in a reduction in the markers and producing a marker-reduced plasma; passing the precipitate and marker-reduced plasma through a filter that captures the precipitate; passing the marker-reduced plasma through a heparin adsorber for removing any residual heparin from the marker-reduced plasma; passing the marker-reduced plasma through a filter for bringing the marker-reduced plasma into a physiological state; recombining the marker-reduced plasma with the formed elements and passing through a filter at a controlled extraction rate to remove excess fluid, resulting in a marker-reduced fluid-depleted blood; and returning the marker-reduced fluid-depleted blood to a vein in the patient.

A method for removing fluid and atherogenic, thrombotic and inflammatory markers from blood includes withdrawing blood from a catheter in a vein of a patient; passing the blood through a filter at a controlled extraction rate to remove excess fluid from the blood, resulting in a fluid-depleted blood; passing the fluid-depleted blood through a plasma filter that separates the fluid-depleted blood into formed elements and plasma; mixing the plasma with a heparin/buffer solution which causes atherogenic, thrombotic, and inflammatory markers in the plasma to form a precipitate, reducing the markers and producing a marker-reduced plasma; passing the precipitate and marker-reduced plasma through a filter that captures the precipitate; passing the marker-reduced plasma through a heparin adsorber for removing any residual heparin from the marker-reduced plasma; passing the marker-reduced plasma through a filter for bringing the marker-reduced plasma into a physiological state; recombining the marker-reduced plasma with the formed elements; and returning the marker-reduced plasma and formed elements to a vein in the patient

Those skilled in the art will recognize that the systems and methods disclosed herein treat other disorders and diseases including, but not limited to, cerebrovascular diseases (including stroke), heart attack, deep vein thrombosis (DVT), retinal artery occlusion (RAO), circulatory disorders, end-stage organ failure, transplants, sepsis, hearing loss, age-related macular degeneration, atherosclerosis, rheumatoid arthritis, autoimmune diseases, Diabetes, Alzheimer's disease, Procoagulant states, dialysis, organ transplant, limb salvage therapy, and neurodegenerative diseases. The systems and methods disclosed herein can be used during open heart surgery while a patient is on a heart bypass machine. The filtration and cannulation process may dislodge athroscloratic material affixed to aorta and dislodge other debris to reduce potential for embolic strokes. The systems and methods disclosed herein may be used for other procedures where a patient shows risk for embolic events.

In an embodiment, the system is an intravascular device delivered to a specific site in the vasculature. For example, and return flow needle with aspiration catheter that is site specific.

In an embodiment, a capillary viscometer, such as the Rheolog capillary viscometer, developed by Rheologics, Inc. (Exton, Pa.), is used before, after, or at any point during the treatment in order to measure the plasma viscosity. In an embodiment, platelet aggregometry is used before, after, or at any point during the treatment to measure erythrocyte aggregation.

All patents, patent applications, and published references cited herein are hereby incorporated by reference in their entirety. It will be appreciated that various of the above-disclosed and other features and functions, or alternatives thereof, may be desirably combined into many other different systems or applications. Various presently unforeseen or unanticipated alternatives, modifications, variations, or improvements therein may be subsequently made by those skilled in the art which are also intended to be encompassed by the following claims.

What is claimed is:

1. A system for removing fluid and atherogenic, thrombotic and inflammatory markers from blood comprising:
an ultrafiltration module for removing fluid from the blood drawn from a patient; and
an apheresis module for removing atherogenic, thrombotic, and inflammatory markers from the blood, wherein the ultrafiltration module and the apheresis module form a continuous closed-loop extracorporeal blood circuit.

2. The system of claim 1 wherein the ultrafiltration module comprises:
a membrane filter for extracting fluid from the blood at a controlled rate;
a collection bag for collecting the removed fluid; and
a roller pump for controlling the flow of fluid through the membrane.

3. The system of claim 1 wherein the apheresis module comprises:
a plasma filter for separating the blood into formed elements and plasma;
a heparin/buffer solution for precipitating out the atherogenic, thrombotic and inflammatory markers from the plasma, yielding a precipitate of markers and marker-reduced plasma;
a filter for capturing the precipitated markers;
a heparin adsorber for removing residual heparin from the marker-reduced plasma; and
a filter for bringing the marker-reduced plasma into a physiological state.

4. The system of claim 1 wherein the atherogenic, thrombotic and inflammatory markers are selected from the group consisting of total cholesterol, low-density lipoprotein cholesterol (LDL-C), Very Low Density Lipoproteins (VLDL), lipoprotein [a] (Lp)[a], triglycerides, fibrinogen, thrombin, Factor V, Factor VII, Von Willebrand factor (vWF), ATP III, C-reactive protein (CRP), plasma viscosity (PV) and erythrocyte aggregation (EA).

5. A method for removing fluid and atherogenic, thrombotic and inflammatory markers from blood comprising:
withdrawing blood through a catheter in a vein of a patient;
passing the blood through a plasma filter that separates the blood into formed elements and plasma;
mixing the plasma with a heparin/buffer solution causing the atherogenic, thrombotic, and inflammatory markers in the plasma to form a precipitate, reducing the markers and producing a marker-reduced plasma;
passing the precipitate and the marker-reduced plasma through a filter that captures the precipitate;
passing the marker-reduced plasma through a heparin adsorber for removing heparin from the marker-reduced plasma;
passing the marker-reduced plasma through a filter for bringing the marker-reduced plasma into a physiological state;
recombining the marker-reduced plasma with the formed elements and passing through a filter at a controlled extraction rate to remove excess fluid, resulting in a marker-reduced fluid-depleted blood; and
returning the marker-reduced fluid-depleted blood to a vein in the patient.

6. The method of claim 5 wherein the atherogenic, thrombotic and inflammatory marker being reduced is selected from the group consisting of total cholesterol, low-density lipoprotein cholesterol (LDL-C), Very Low Density Lipoproteins (VLDL), lipoprotein [a] (Lp)[a], triglycerides, fibrinogen, thrombin, Factor V, Factor VII, Von Willebrand factor (vWF), ATP III, C-reactive protein (CRP), plasma viscosity (PV) and erythrocyte aggregation (EA).

7. The method of claim 5 wherein the heparin/buffer solution has a plasma reaction pH of about 5.12.

8. The method of claim 5 wherein bicarbonate dialysis and ultrafiltration bring the marker-reduced plasma into a physiological state.

9. The method of claim 5 wherein a roller pump passes the marker-reduced plasma and the formed elements through the ultra-filter at a controlled rate.

10. The method of claim 6 wherein the marker being reduced is plasma viscosity.

11. The method of claim 10 wherein the reduction in plasma viscosity and the removal of excess fluid an improves the symptoms of congestive heart failure.

12. A method for removing fluid and atherogenic, thrombotic and inflammatory markers from blood comprising:
withdrawing blood from a vein of a patient through a catheter;
passing the blood through a filter at a controlled extraction rate to remove fluid from the blood, resulting in a fluid-depleted blood;
passing the fluid-depleted blood through a plasma filter to separate the fluid-depleted blood into formed elements and plasma;
mixing the plasma with a heparin/buffer solution causing atherogenic, thrombotic, and inflammatory markers in the plasma to form a precipitate, reducing the markers and producing a marker-reduced plasma;
passing the precipitate and the marker-reduced plasma through a filter to capture the precipitate;
passing the marker-reduced plasma through a heparin adsorber for removing heparin from the marker-reduced plasma;
passing the marker-reduced plasma through a filter to bring the marker-reduced plasma into a physiological state;
recombining the marker-reduced plasma with the formed elements; and
returning the marker-reduced plasma and formed elements to a vein in the patient.

13. The method of claim 12 wherein the atherogenic, thrombotic and inflammatory marker being reduced is selected from the group consisting of total cholesterol, low-density lipoprotein cholesterol (LDL-C), Very Low Density Lipoproteins (VLDL), lipoprotein [a] (Lp)[a], triglycerides, fibrinogen, thrombin, Factor V, Factor VII, Von Willebrand factor (vWF), ATP III, C-reactive protein (CRP), plasma viscosity (PV) and erythrocyte aggregation (EA).

14. The method of claim 12 wherein the heparin/buffer solution has a plasma reaction pH of about 5.12.

15. The method of claim 12 wherein bicarbonate dialysis and ultrafiltration bring the marker-reduced plasma into a physiological state.

16. The method of claim 12 wherein a roller pump passes the marker-reduced plasma and the formed elements through the ultra-filter at a controlled rate.

17. The method of claim 13 wherein the marker being reduced is plasma viscosity.

18. The method of claim 17 wherein the reduction in plasma viscosity and the removal of excess fluid improves the symptoms of congestive heart failure.
